(19) United States
(12) Patent Application Publication
(10) Pub. No.: US 2012/0003689 A1
(43) Pub. Date: Jan. 5, 2012

(54) HYDROPHOBIC MAGNETIC PARTICLES

(75) Inventors: Jackie Y. Ying, Singapore (SG); Su Seong Lee, Singapore (SG); Siti Nurhanna Binte Riduan, Singapore (SG); yu Han, Singapore (SG)

(73) Assignee: Agency for Science, Technology and Research, Singapore (SG)

(21) Appl. No.: 13/129,388

(22) PCT Filed: Nov. 17, 2008
(86) PCT No.: PCT/SG08/00435

§ 371 Date: May 13, 2011

(51) Int. Cl. C12N 11/14; H01F 1/01; H01F 1/36; B05D 3/00; C12P 1/00; B05D 7/00; B05D 5/00; B05D 3/02; C07C 67/03; H01F 1/33

(52) U.S. Cl. 435/41; 560/254; 252/62.51 R; 252/62.56; 252/62.59; 435/176; 427/212; 427/130

(57) ABSTRACT

A process for making a particulate material comprising mesoporous particles having granules of a metal containing species in at least some of the pores thereof, said process comprising: allowing a compound of the metal to enter pores of hydrophobic mesoporous particles, said compound being thermally decomposable at a decomposition temperature to form a metal containing species and said particles being substantially thermally stable at said decomposition temperature; and heating the hydrophobic mesoporous particles having the compound in the pores thereof to the decomposition temperature so as to decompose the compound and to form the mesoporous particles having granules of the metal containing species in at least some of the pores thereof.

### TECHNICAL FIELD

The present invention relates to hydrophobic magnetic particles, their synthesis and their use.

### BACKGROUND OF THE INVENTION

Enzyme-catalyzed biotransformation have gained increasing importance in organic and pharmaceutical syntheses. This is due to the superior specificity and selectivity of certain enzymes, and the milder reaction conditions employed compared to conventional chemical synthesis of enantioselective compounds.

The main challenges facing the scale-up of enzyme-catalyzed asymmetric reactions include the difficulty of enzyme separation and recycling from the solution, and the deactivation of enzymes when organic solvents are used. To overcome these issues, enzymes have been immobilized onto solid supports to render them more mechanically robust and thermally stable, and to facilitate catalyst recovery and reuse. Immobilized enzymes have also been reported to exhibit enhanced catalytic activity due to reduced enzyme aggregation in organic media and less protein denaturation [(a) Lalonde, J.; Margolin, A. Enzyme Catalysis in Organic Synthesis; Wiley-VCH: Weinheim, Germany, 2002; Vol. 2, p. 163. (b) Yiu, H. H. P.; Wright, P. A. J. Mater. Chem. 2005, 15, 3690. (c) Maury, S.; Buisson, P.; Pierre, A. C. J. Mol. Catal. B: Enzym. 2002, 19, 269].

There are generally three ways to immobilize enzymes onto solid inorganic supports: encapsulation, covalent bonding and entrapment. However, the separation of such catalysts from a reaction mixture would generally require high-speed centrifugation. There is a need to develop catalytic materials with open, interconnected and ultralarge pores to facilitate substrate diffusion to catalytic sites, while incorporating attributes that would allow for the easy separation of the catalytic material from the reaction mixture.

Methods of incorporating the magnetic characteristics in mesoporous silica have been reported recently. They include the electrochemical synthesis of magnetic nanoparticles within the walls of the silica supports, and the synthesis of γ-Fe₂O₃ within SBA-15, MCM-41 and MCM-48 channels via impregnation and exposure to organic acids. However, such synthesis methods commonly lead to pore blocking. To overcome this problem, Lu and his co-workers fabricated magnetically separable mesostructured silica with an open pore system, whereby the magnetic cobalt nanoparticles were selectively deposited on the surface of SBA-15 support (Lu, A.-H.; Li, W.-C.; Kiefer, A.; Schmidt, W.; Bill, E.; Fink, G.; Schüth, F. J. Am. Chem. Soc. 2004, 126, 8616). This process retained the pore structure, but it was tedious and unclear if the cobalt nanoparticles would detach from the SBA-15 particles after frequent use. γ-Fe₂O₃ has also been grafted onto the mesopore surface of HMMS (hierarchically ordered mesocellular mesoporous silica), and the use of the resulting support in immobilizing ligands for asymmetric dihydroxylation reactions have been reported (Lee, D.; Lee, J.; Lee, H.; Jin, S.; Hyeon, T.; Kim, B. M. Adv. Synth. Catal. 2006, 348, 41). Magnetic nanoparticles of 6 nm have also been entrapped in HMMS by the crosslinking of enzymes with the nanoparticles using glutaraldehyde (Kim, J.; Lee, J.; Na, H. B.; Kim, B. C.; Youn, J. K.; Kwak, K. M., Lee, E.; Kim, J.; Park, J.; Dohnalkova, A.; Park, H. G.; Gu, M. B.; Chang, H. N.; Grate, J. W.; Hyeon, T. Small 2005, 1, 1203).

There is therefore a need for a convenient process for producing magnetic particles having catalytic properties. A suitable process would preferably be relatively rapid and simple to implement.

### OBJECT OF THE INVENTION

It is the object of the present invention to substantially overcome or at least ameliorate one or more of the above disadvantages. It is a further object to at least partially satisfy at least one of the above needs.

### SUMMARY OF THE INVENTION

In a broad form of the invention there is provided a process for making a particulate material comprising mesoporous particles having granules of a metal containing species in at least some of the pores thereof, said process comprising:
allowing a compound of the metal to enter pores of hydrophobic mesoporous particles, said compound being thermally decomposable at a decomposition temperature to form a metal containing species and said particles being substantially thermally stable at said decomposition temperature; and
heating the hydrophobic mesoporous particles having the compound in the pores thereof to the decomposition temperature so as to decompose the compound and to form the mesoporous particles having granules of the metal containing species in at least some of the pores thereof; and
optionally rendering the pore surfaces of the hydrophobic mesoporous hydrophilic.

The metal may be iron, or it may be some other metal. It may be a transition metal. It may be cobalt. It may be platinum. It may be palladium. It may be a combination of more than one, for example 2 or 3, metals (e.g. transition metals). The granules may be magnetic. They may be non-magnetic. They may be ferromagnetic. They may be paramagnetic. They may comprise an oxide of the metal or oxides of the metals. In one embodiment the metal is iron and the metal containing species comprises iron oxide.

In a first aspect of the invention there is provided a process for making a particulate material comprising hydrophobic magnetic particles, said process comprising:
allowing an iron compound to enter pores of hydrophobic mesoporous particles, said iron compound being thermally decomposable at a decomposition temperature and said particles being substantially thermally stable at said decomposition temperature; and
heating the hydrophobic mesoporous particles having the iron compound in the pores thereof to the decomposition temperature so as to decompose the iron compound and to form the hydrophobic magnetic particles having magnetic granules in at least some of the pores.

The following options may be used in conjunction with the first aspect, either individually or in any suitable combination.

The process may comprise the step of allowing a facilitation agent to enter the pores of the hydrophobic mesoporous particles prior to allowing the iron compound to enter said pores. The facilitation agent may be a compound capable of facilitating or accelerating the high temperature decomposition of the iron compound. It may be a compound capable of stabilising the granules. It may be a surfactant. It may be a carboxylic acid. It may be an alkanoic acid or an alkenoic acid.

The iron compound may be an iron carbonyl complex. It may be iron pentacarbonyl. The magnetic granules may comprise magnetic γ-Fe₂O₃. The magnetic granules may be substantially spherical.

The decomposition temperature may be about 250 to about 350° C.

The process may comprise the additional step of cooling the hydrophobic magnetic particles. It may comprise the additional step of treating the cooled hydrophobic magnetic particles with a decomposing agent, e.g. an oxidising agent. In the event that the iron compound is iron pentacarbonyl, the oxidising agent may be an amine oxide, for example trimethylamine N-oxide. The step of treating the cooled hydrophobic magnetic particles with the oxidising agent may comprise heating the hydrophobic magnetic particles with the oxidising agent.

The hydrophobic mesoporous particles may be hydrophobic mesoporous silica. The process may comprise reacting mesoporous silica particles with a hydrophobing agent so as to produce the hydrophobic mesoporous particles. The surfaces of the pores of the hydrophobic mesoporous particles may comprise hydrophobic groups such as trialkylsilyl groups, e.g. trimethylsilyl groups, dimethyloctylsilyl groups, dimethyloctadecylsilyl groups or a mixture of any two or all of these.

The process may comprise the step of immobilising a catalytic species in the pores of the hydrophobic magnetic particles. The catalytic species may be an enzyme. The step of immobilising the enzyme may comprise passing a fluid comprising the enzyme through the hydrophobic magnetic particles under high pressure. The pressure may be at least about 25 MPa. It may be between about 25 and about 50 MPa. The fluid may be an aqueous liquid. In some cases the immobilising may comprise rendering the pore surfaces of the hydrophobic magnetic particles hydrophilic and then immobilising the catalytic species on the hydrophilic pore surfaces. In some cases the step of rendering the pore surfaces hydrophilic may comprise removing hydrophobic groups from the pore surfaces.

In an embodiment there is provided a process for making a particulate material comprising hydrophobic magnetic particles, said process comprising:
allowing iron pentacarbonyl to enter pores of hydrophobic mesoporous silica particles; and
heating the hydrophobic mesoporous particles having the iron compound in the pores thereof to about 250 to about 350° C. so as to decompose the iron pentacarbonyl and to form the hydrophobic magnetic particles having magnetic granules in at least some of the pores.

In another embodiment there is provided a process for making a particulate material comprising hydrophobic magnetic particles, said process comprising:
allowing a facilitation agent to enter the pores of mesoporous silica particles;
allowing iron pentacarbonyl to enter the pores; and
heating the hydrophobic mesoporous particles having the iron compound in the pores thereof to about 250 to about 350° C. so as to decompose the iron pentacarbonyl and to form the hydrophobic magnetic particles having magnetic granules in at least some of the pores.

In another embodiment there is provided a process for making a particulate material comprising hydrophobic magnetic particles, said process comprising:
treating mesoporous silica with a hydrophobing agent so as to produce mesoporous silica particles;
allowing a carboxylic acid facilitation agent to enter the pores of the mesoporous silica particles;
allowing iron pentacarbonyl to enter the pores of the mesoporous silica particles;
heating the hydrophobic mesoporous particles having the iron compound in the pores thereof to about 250 to about 350° C. so as to decompose the iron pentacarbonyl and to form the hydrophobic magnetic particles having magnetic granules in at least some of the pores;
cooling the hydrophobic magnetic particles;
treating the cooled hydrophobic magnetic particles with trimethylamine N-oxide;
washing the resulting hydrophobic magnetic particles; and
passing a fluid comprising an enzyme through the hydrophobic magnetic particles a pressure of at least about 25 MPa so as to immobilise the enzyme in the pores of the hydrophobic magnetic particles.

In a second aspect of the invention there is provided a particulate material comprising a plurality of magnetic particles, said particles comprising mesoporous particles having magnetic granules in at least some of the pores thereof. The magnetic particles and the mesoporous particles may both be hydrophobic.

More broadly there is provided a particulate material comprising a plurality of particles, said particles comprising mesoporous particles having granules of a metal containing species in at least some of the pores thereof. The metal containing species may be a metal. It may be a metal oxide. It may be some other metal containing substance. It is preferably a solid. The metal may be iron or cobalt or platinum or palladium. It may be a transition metal or a mixture of transition metals.

The following options may be used in conjunction with the second aspect, either individually or in any suitable combination.

The mesoporous particles may be hydrophobic mesoporous silica particles. The pores may have surfaces comprising hydrophobic groups such as trialkylsilyl groups.

The mesoporous particles may have a structure comprising pores connected by windows, wherein the mean diameter of the windows is smaller than the mean diameter of the pores. The magnetic granules may have a mean diameter between the mean diameter of the pores and the mean diameter of the windows.

The magnetic granules may comprise magnetic γ-Fe₂O₃. They may be substantially spherical.

The particulate material may have a catalytic species immobilised in the pores of the particles. The catalytic species may be an enzyme. The diameter of the enzyme may be between the mean diameter of the pores and the mean diameter of the windows.

In an embodiment there is provided a particulate material comprising a plurality of hydrophobic magnetic particles, said particles comprising hydrophobic mesoporous particles having magnetic γ-Fe₂O₃ granules in at least some of the pores thereof.

In another embodiment there is provided a particulate material comprising a plurality of hydrophobic magnetic particles, said particles comprising hydrophobic mesoporous silica particles, pores of said particles having surfaces comprising trialkylsilyl groups and said particles having magnetic γ-Fe₂O₃ granules in at least some of the pores thereof.

In another embodiment there is provided a particulate material comprising a plurality of hydrophobic magnetic particles, said particles comprising hydrophobic mesoporous silica particles, pores of said particles having surfaces comprising trialkylsilyl groups and said particles having magnetic γ-Fe₂O₃ granules in at least some of the pores thereof and having an enzyme immobilised in the pores thereof.

In another embodiment there is provided a particulate material comprising a plurality of magnetic particles, said particles comprising mesoporous particles having magnetic granules in at least some of the pores thereof and the pore walls thereof having an enzyme immobilised thereon.

In a third aspect of the invention there is provided use of a particulate material as a catalyst, said particulate material being as described in the second aspect and said particulate material having a catalytic species immobilised in the pores of the particles. The invention also provides a particulate material when used as a catalyst, said particulate material being as described in the second aspect and said particulate material having a catalytic species immobilised in the pores of the particles.

In a fourth aspect of the invention there is provided a method for converting a starting material to a product, said method comprising exposing the starting material to a particulate material comprising a plurality of magnetic particles, said particles comprising mesoporous particles having magnetic granules in at least some of the pores thereof and having a catalytic species immobilised in the pores of the particles, wherein the catalytic species is capable of catalysing the conversion of the starting material to the product. The magnetic particles and the mesoporous particles may both be hydrophobic.

The following options may be used in conjunction with the fourth aspect, either individually or in any suitable combination.

The starting material may be dissolved in a solvent during the step of exposing said starting material to said particulate material. The solvent may be a non-polar solvent. The step of exposing may comprise adding the particulate material to a solution of the starting material in the solvent.

The method may comprise the step of separating the particulate material from the starting material and the product. The step of separating may comprise exposing the particulate material to a magnetic field. The method may also comprise the step of reusing the particulate material as a catalyst in a subsequent reaction. The subsequent reaction may convert the starting material to the product. The yield of the product of the subsequent reaction may be at least 90% of the yield of the product from the previous reaction.

In an embodiment there is provided a method for converting a starting material to a product, said method comprising adding a particulate material to a solution of the starting material in a non-polar solvent, said particulate material comprising a plurality of hydrophobic magnetic particles, said particles comprising hydrophobic mesoporous particles having magnetic granules in at least some of the pores thereof and having a catalytic species immobilised in the pores of the particles, wherein the catalytic species is capable of catalysing the conversion of the starting material to the product.

In another embodiment there is provided a method for converting a starting material to a product, said method comprising:
adding a particulate material to a solution of the starting material in a non-polar solvent, said particulate material comprising a plurality of hydrophobic magnetic particles, said particles comprising hydrophobic mesoporous particles having magnetic granules in at least some of the pores thereof and having a catalytic species immobilised in the pores of the particles, wherein the catalytic species is capable of catalysing the conversion of the starting material to the product;
exposing the particulate material to a magnetic field so as to separate the particulate material from the product and, if present, the starting material; and
reusing the particulate material as a catalyst in a subsequent reaction;
   whereby the yield of the product of the subsequent reaction is at least 90% of the yield of the product from the previous reaction.

### BRIEF DESCRIPTION OF THE DRAWINGS

Preferred embodiments of the present invention will now be described, by way of example only, with reference to the accompanying drawings wherein:

FIG. 1 shows SEM images of catalyst D supported on γ-Fe₂O₃/C₁₈-MCF microparticles;

FIG. 2 shows TEM images of catalyst D supported on γ-Fe₂O₃/C₁₈-MCF microparticles;

FIG. 3 shows a powder XRD pattern of γ-Fe₂O₃/C₁₈-MCF microparticles;

FIG. 4 shows nitrogen adsorption-desorption isotherms of (□) C₁₈-MCF, (x) γ-Fe₂O₃/C₁₈-MCF, and (Δ) catalyst D;

FIG. 5 shows PA-FTIR spectra of γ-Fe₂O₃/C₈-MCF (a) before and (b) after enzyme entrapment;

FIG. 6 shows a graph of conversion of 1-phenylethanol as a function of time over (♦) catalyst A, (▪) catalyst B, (▴) catalyst C and () catalyst D, and (∘) free CALB;

FIG. 7 is Nitrogen adsorption-desorption isotherms of (□) catalyst A, (Δ) catalyst B, (x) catalyst C, and (∘) catalyst D; and

FIG. 8 shows a graph of conversion of 1-phenylethanol as a function of time for Catalyst D over 5 consecutive runs.

### DETAILED DESCRIPTION OF THE INVENTION

An embodiment of the present invention relates to the synthesis of novel magnetic, hydrophobic, siliceous mesocellular foam (MCF) and its application in enzyme immobilization. In the present specification, the term “magnetic” is taken to refer to a material that is “capable of being magnetised or attracted by a magnet” (definition from the Macquarie Dictionary, 2nd Edition, The Macquarie Library Pty Ltd 1991). Many enzymatic catalysts have been immobilized on siliceous and other inorganic supports. Although they are more mechanically robust and thermally stable than polymer-supported catalysts, the separation of such catalysts from the reaction mixture generally requires high-speed centrifugation or filtration. Such procedures are tedious and difficult to apply towards large-scale synthesis of pharmaceuticals and fine chemicals. The present inventors have successfully introduced magnetic attributes to the hydrophobic MCF support to facilitate the recovery and reuse of enzymatic catalysts. This novel support material can also be applied towards immobilizing organometallic and organic catalysts to enhance catalyst separation. The catalysts supported on magnetic MCF are robust and easily collected, by means of a magnet, for reuse. The presently described synthesis of magnetic MCF is novel, as the magnetic granules are entrapped in the mesopores, and the resulting material offers a well-defined, three-dimensional porous structure.

This invention provides a facile and inexpensive method for the preparation of magnetic MCF supports for use as efficient and reusable catalyst systems. In particular, the introduction of magnetic attributes does not disrupt the three-dimensional porosity of mesoporous silica. The magnetic MCF may also be tailored easily in terms of microstructure, pore size, surface chemistry and magnetic granule loading for particular applications. The magnetic granules may be magnetic nanoparticles.

The present invention describes a process for making a particulate material comprising hydrophobic magnetic particles. In the process an iron compound is passed into the pores of hydrophobic mesoporous particles. The hydrophobic mesoporous particles having the iron compound in the pores are then heated to the decomposition temperature of the iron compound so as to decompose the iron compound so as to form magnetic granules in at least some of the pores. The decomposition of the iron compound may be facilitated by a facilitation agent.

The step of passing the iron compound into the pores may be conducted using gas phase or liquid phase iron compound. The nature of this may depend on the nature, e.g. volatility, of the iron compound. It may be passed into the pores by passing a vapour or a liquid comprising the iron compound into the pores. This may comprise locating the particles in the vapour or liquid, or it may comprise passing the vapour or liquid past or through said particles. It may comprise forming a bed of the particles and passing the vapour or liquid through the bed. It may comprise passing a vapour into the particles. The vapour or liquid may comprise a carrier. In the case of a vapour this may comprise an inert gas e.g. nitrogen, carbon dioxide, argon, helium etc. In the case of a liquid it may comprise a solvent for the iron compound that does not react with the iron compound. In a particular embodiment, the iron compound is added as a neat liquid. The iron compound may be used at a ratio of about 1 to about 5 mmol per gram of hydrophobic particles, or about 1 to 3, 3 to 5, 2 to 4 or 2 to 3 mmol/g, e.g. about 1, 1.5, 2, 2.5, 3, 3.5, 4, 4.5 or 5 mmol/g.

The step of heating the hydrophobic mesoporous particles having the iron compound in the pores so as to decompose the iron compound may optionally be conducted in the presence of a decomposing agent capable of decomposing the iron compound. In the event the iron compound is iron pentacarbonyl, the decomposing compound may be an amine oxide e.g. trimethylamine N-oxide. In some cases the decomposing agent is added after the formation of the majority of the magnetic particles have formed, commonly after the particles have been at least partially cooled. The heating may be to a suitable decomposition temperature. It may be between about 100 and about 400° C., or about 100 to 300, 100 to 200, 200 to 400, 300 to 400 or 250 to 350° C., e.g. about 100, 150, 200, 250, 300, 350 or 400° C. The heating may be in different stages, e.g. 2 or 3 stages, each of which may independently be at one of the temperatures or ranges above, or the heating may be in a single stage. The temperature may depend on the nature of the iron compound, and may also depend on the presence or absence of a decomposing agent.

The inventors have found in one example of the process of the invention that iron pentacarbonyl:oleic acid complex in the pores of mesoporous silica particles is decomposed at about 300° C. The complexes start to form at a temperature above about 100° C. The initial decomposition of the complex leads to the formation of seed particles/granules. The seed particles grow during heating for 1 hr at 300° C. The inventors is have found that they were able to control the amount of entrapped magnetic granules by adjusting the amount of iron pentacarbonyl and oleic acid used in the process. Different catalysts may be prepared having different amounts of entrapped magnetic granules and different pore size of MCF. It is also possible to entrap other metals such into the pores of the mesoporous silica having cage-like pores, which may be used as catalysts. The inventors find that it is preferable to use surfactants with a short carbon chain to entrap surfactant-stabilized nanoparticles in the pores of small pore size particles. To entrap magnetic nanoparticles in the small pores of FDU-12 (5 nm window pore, 8 nm cell pore), hexanoic acid was used instead of oleic acid. Other than when FDU-12 was used, magnetic granules were commonly formed using oleic acid.

The hydrophobic mesoporous particles may comprise silica, or a metal, or a metal oxide or mixed metal oxide. The metal may be for example iron, titanium, zirconium or aluminium. The hydrophobic mesoporous particles may be a foam, for example open celled foam, or may be sintered or otherwise porous. It may be mesostructured cellular foam (MCF) or FDU-12, as described in Schmidt-Winkel et al, Science, 1999, 548, Lettow et al, Langmuir, 2000, 16, 8291 and Fan et al, Angew. Chem. Int. Ed., 2003, 42, 3146. It may be a silica foam according to PCT application PCT/SG2005/000194, the contents of which are incorporated herein by cross reference. The hydrophobic mesoporous particles may have a particle size between about 100 nm and 200 microns. The particle size may be between about 500 nm and 200 microns, or between about 1 and 200, 10 and 200, 50 and 200, 100 and 200, 1 and 100, 1 and 50 or 1 and 10 microns or between about 100 nm and 100 microns, 100 nm and 10 microns, 100 nm and 1 micron or 500 nm and 1 micron, and may be about 100, 200, 300, 400, 500, 600, 700, 800 or 900 microns, or about 1, 2, 3, 4, 5, 6, 7, 8, 9, 10, 20, 30, 40, 50, 60, 70, 80, 90, 100, 110, 120, 130, 140, 150, 160, 170, 180, 190 or 200 microns. The hydrophobic mesoporous particles may have a narrow particle size distribution. There may be less than about 50% of particles having a particle size more than 10% different from (greater than or less than) the mean particle size, or there may be less than about 45, 40, 35, 30, 25, 20, 15, 10 or 5% of particles having a particle size more than 10% different from the mean particle size, and may be about 1, 2, 3, 4, 5, 10, 15, 20, 25, 30, 35, 40, 45 or 50% of particles having a particle size more than 10% different from the mean particle size. The hydrophobic mesoporous particles may comprise cell-like mesopores connected by windows of a smaller size. The ratio of the size of the mesopores and the size of the windows may be between about 10:1 and 1.5:1, or between about 10:1 and 2:1, 10:1 and 5:1, 5:1 and 1.5:1, is 3:1 and 1.5:1, 5:1 and 3:1 or 8:1 and 4:1, and may be about 10:1, 9:1, 8:1, 7:1, 6:1, 5:1, 4.5:1, 4:1, 3.5:1, 3:1, 2.5:1, 2:1 or 1.5:1, or may be some other ratio. It should be understood that when reference is made to the “pore size” of such materials, it refers to the effective pore size, i.e. the size of the narrowest portion of a flow channel through the material. Thus in a structure comprising cell-like mesopores connected by windows of a smaller size, the “pore size” refers to the size of the windows, and not to the size of the mesopores. The mesoporous particles may have a mean pore size (i.e. window diameter) of between about 2 and 50 nm or between about 2 and 20, 2 and 10, 5 and 20, 5 and 10, 10 and 40, 10 and 30, 10 and 20, 20 and 50, 30 and 50, 40 and 50, 20 and 40 or 20 and 30 nm, and may have a mean pore size about 2, 3, 4, 5, 6, 7, 8, 9, 10, 11, 12, 13, 14, 15, 20, 25, 30, 35, 40, 45 or 50 nm. The actual pores may have a mean diameter of about 10 to about 100 nm, or 10 to 50, 10 to 20, 20 to 100, 50 to 100 or 20 to 50 nm, e.g. about 10, 11, 12, 13, 14, 15, 16, 17, 18, 19, 20, 25, 30, 35, 40, 45, 50, 60, 70, 80, 90 or 100 nm. The hydrophobic mesoporous particles may have a pore volume between about 0.5 and 5 cm³/g, and may have a pore volume between about 0.5 and 2, 0.5 and 1, 1 and 5, 3 and 5 or 1 and 3 cm³/g, and may have a pore volume between about 0.5, 1, 1.5, 2, 2.5, 3, 3.5, 4, 4.5 or 5 cm³/g. They may have a void volume of between about 50 and 90%, or between about 50 and 70, 60 and 70, 70 and 80, 80 and 90 or 75 and 85%, and may have a void volume of about 50, 55, 60, 65, 70, 75, 80, 85 or 90%. They may have a bulk density of between about 0.2 and 1 g/ml, or between about 0.5 and 1, 0.2 and 0.5, 0.2 and 0.4, 0.2 and 0.3, 0.3 and 0.4 or 0.25 and 0.35 g/ml. and may have a bulk density of about 0.2, 0.25, 0.3, 0.35, 0.4, 0.45, 0.5, 0.55, 0.6, 0.65, 0.7, 0.75, 0.8, 0.85, 0.9, 0.95 or 1 g/ml.

The process described herein may comprise the step of hydrophobing mesoporous particles to obtain the hydrophobic mesoporous particles. The step of hydrophobing may comprise exposing the particles to a hydrophobing agent. The hydrophobing agent may be in solution. It may be dissolved in a solvent. The hydrophobing agent may have a group capable of reacting with the porous support, and may also have at least one hydrophobic group. For example, if the precursor particles comprise silica, then the hydrophobing agent may comprise a hydrolysable group, such as a chlorosilyl group, an alkoxysilyl group, a silazane group or some other suitable group. The hydrophobic agent may be a silane, for example a halosilane, a silazane or an alkoxysilane or some other type of hydrolysable silane (such as an acetoxysilane, an oximosilane, an amidosilane etc.). The hydrophobic group may be an alkyl group, for example C1 to C24 alkyl or bigger than C24 alkyl, or an aryl group, for example C6 to C12 aryl, or some other suitable is hydrophobic group. The alkyl group may be straight chain or branched chain, and may have between 1 and 24 carbon atoms, or between 1 and 18, 1 and 12, 1 and 6, 6 and 24, 12 and 24 or 6 and 18 carbon atoms, and may have 1, 2, 3, 4, 5, 6, 7, 8, 9, 10, 11, 12, 14, 16, 18, 20, 22 or 24 carbon atoms. It may comprise a cycloalkyl group such as cyclopentyl, cyclohexyl or cycloheptyl. The aryl group may be for example phenyl, biphenyl, naphthyl or some other aryl group. The aryl or alkyl group may be fluorinated or polyfluorinated or perfluorinated. The hydrophobing agent may have one, two, three or more than three hydrophobic groups per molecule. It may for example have a formula RₙSiX₄₋ₙ or RMe₂SiCl, where R is the hydrophobic group, X is the hydrolysable group and n is 1, 2 or 3. Alternatively the hydrophobing agent may comprise a siloxane or a cyclosiloxane. Suitable hydrophobing agents may include chlorodimethyloctylsilane, chlorodimethyloctadecylsilane, methoxytrimethylsilane, dimethyldimethoxysilane, hexamethyldisilazane, hexamethyldisiloxane, decamethylcyclopentasiloxane (D5) or other cyclosiloxanes. The process of hydrophobing may comprise exposing the precursor particles to the hydrophobing agent, optionally together with a catalyst, for between about 1 and 48 hours, for example between 1 and 24, 1 and 12, 12 and 48, 24 and 48 or 12 and 36 hours (e.g. for about 1, 2, 3, 4, 5, 6, 12, 18, 24, 30, 36, 42 or 48 hours) at a temperature between about 10 and 80° C. The temperature may be between about 10 and 60, 10 and 40, 10 and 20, 20 and 80, 40 and 80, 60 and 80, 20 and 60 or 40 and 60° C., and may be about 10, 20, 30, 40, 50, 60, 70 or 80° C. The catalyst may depend on the nature of the hydrophobing agent and of the precursor particles. It may be for example an amine, such as a tertiary amine, and may be for example trimethylamine or triethylamine, pyridine or some other base. The hydrophobing agent and, if present, the catalyst, may be dissolved in a solvent. The solvent may be organic, and may be non-hydroxylic, and may be for example toluene, xylene or some other suitable solvent. The exposing may comprise immersing the precursor particles in a solution of the hydrophobing agent in the solvent, and may comprise stirring, swirling, shaking, sonicating or otherwise agitating the solution with the porous support therein, or it may comprise passing the solution through the porous support, and optionally recirculating the solution through the porous support. It may be a vapour phase or gas phase hydrophobising reaction.

The precursor particles may be degassed and/or dried before being hydrophobed. They may be heated to a temperature between about 100 and 200° C., for example between 100 and 150, 100 and 120, 150 and 200, 170 and 200 or 125 and 175° C. (e.g. about 100, 110, 120, 130, 140, 150, 160, 170, 180, 190 or 200° C.). It may have heated, and optionally is dried, gases passed therethrough at a temperature as listed above. They may have gases, optionally dried gases, passed therethrough at atmospheric temperature. It may be exposed to a vacuum at a temperature as listed above. The vacuum may have an absolute pressure of less than about 10⁻² torr, or less than about 5*10⁻³, 10⁻³, 5*10⁻⁴, 10⁴, 5*10⁻⁵, 10⁻⁵, 5*10⁻⁶ or 10⁻⁶ torr, and may have an absolute pressure of between about 10⁻² and 10⁻⁶ torr, or between about 10⁻³ and 10⁻⁶ torr, 10⁻⁴ and 10⁻⁶ torr, 10⁻⁵ and 10⁻⁶ torr, 10⁻³ and 10⁻⁵ torr or 10⁻⁴ and 10⁻⁵ torr, and may have a pressure of about 5*10⁻³, 10⁻³, 5*10⁻⁴, 10⁻⁴, 5*10⁻⁵, 10⁻⁵, 5*10⁻⁶ or 10⁻⁶ torr.

After being hydrophobed, the hydrophobic mesoporous particles may be washed one or more times. Each wash may be with a different washing solvent, or some of the washes may be with the same solvent. The solvent may be aqueous or may be organic. The organic solvent may be polar or non-polar. Suitable solvents include water, methanol, ethanol, isopropanol, acetone, dichloromethane, ethyl acetate, toluene and xylene, and may also be any miscible combination of suitable solvents. After any or all of the washes the porous support may be dried. The drying may comprise for example heating (for example as described above), passing a gas through the hydrophobic mesoporous particles, or exposing them to a vacuum (for example as described above). The gas may be air, nitrogen, carbon dioxide or some other gas, and may be heated or may be not heated.

The process of the present invention may comprise the step of allowing a facilitation agent to enter the pores of the hydrophobic mesoporous particles prior to allowing the iron compound to enter said pores. The facilitation agent may be capable of facilitating decomposition of the iron compound to form the granules. It may be capable of facilitating the decomposition at elevated temperature. It may be capable of reacting, or associating, or complexing, with the iron compound so as to facilitate the decomposition. It may form an iron compound-facilitation agent complex, which subsequently decomposes at high temperature to form the granules. The facilitation agent may be capable of stabilising the granules formed in the pores of the particles. The facilitation agent may be a surfactant. It may be an anionic surfactant. It may be a cationic surfactant. It may be a non-ionic surfactant. It may be a zwitterionic surfactant. In some cases more than one facilitation agent, or more than one type of facilitation agent, may be used. The facilitation agent may be for example a long chain fatty acid. It may be saturated or it may be unsaturated. It may be for example a C6 to C20 saturated or unsaturated fatty acid, or is C6 to C18, C6 to C12, C12 to C18, C14 to C20 or C24 to C18. It may for example be oleic acid. It may be hexanoic acid. It may be a mixture of fatty acids. It may be a mixture of C12 to C20 fatty acids, any one or more of which may be unsaturated. The length of the chain of the carboxylic acid may depend on the pore size of the mesoporous particles. The facilitation agent may enter the pores in solution. The solvent for the solution may be a volatile solvent or a slightly volatile solvent. It may be an ether or a ketone or some other suitable solvent. It may be for example dioctyl ether. The solution may be about 10 to about 30% in the surfactant, or about 10 to 20, 20 to 30 or 15 to 25%. The step of allowing the surfactant to enter the pores may comprise immersing the hydrophobic mesoporous particles in the solution, or it may comprise passing the solution through and/or past the particles. It may comprise forming a bed of the particles and passing the solution through the bed. The passing may be under gravity or it may be under an applied pressure. The applied pressure may be sufficient to cause the solution to enter the pores of the particles. The solution may be dried following entering the pores. This may comprise heating the particles having the solution in the pores thereof. It may comprise applying a vacuum to the particles having the solution in the pores thereof. It may comprise both of these. The temperature and pressure and time may be sufficient to remove substantially all of the moisture in the solution. These three factors will of course be interdependent, so that, for example, the lower the pressure, the shorter the time may be, and will also depend on the nature (in particular the volatility) of the solvent and of the surfactant. These factors should preferably be sufficient to remove substantially all of the moisture but not remove substantial amounts of the surfactant. Suitable temperatures are generally about 30 to about 80° C., or about 30 to 50, 50 to 80 or 40 to 60° C., e.g. about 30, 40, 50, 60, 70 or 80° C. The pressure may be about 10⁻¹ mbar to about 10⁻² mbar. The time required may be about 5 to about 30 hours, or about 5 to 20, 10 to 30 or 20 to 30 hours, e.g. about 5, 10, 15, 20, 25 or 30 hours. In some instances this step may be omitted, or may take less time, e.g. 1, 2, 3 or 4 hours.

As discussed earlier, the iron compound may be an iron carbonyl complex. It may be iron pentacarbonyl. It may be some other iron compound provided that it is stable in to organic solution at room temperature and is decomposable at elevated temperature, optionally in the presence of a facilitation agent as described elsewhere herein. The magnetic granules may comprise iron oxide, for example magnetic γ-Fe₂O₃. They may be substantially spherical. They may not be elongated. They may not be magnetic wires. They may be constrained within the pores of the particles. They may be of a suitable shape that they do not substantially impede flow of a fluid through the pores and through the windows joining the pores. They may have a size that is intermediate between that of the windows of the particles and the pores of the particles so that they are able to fit in the pores but not pass out of them through the windows.

The process may comprise the additional step of cooling the hydrophobic magnetic particles. The cooling may be to a temperature of between about 50 to about 150° C., or about 50 to 100, 100 to 150 or 70 to 120° C., e.g. about 50, 60, 70, 80, 90, 100, 110, 120, 130, 140 or 150° C. It may comprise the additional step of treating the cooled hydrophobic magnetic particles with a decomposing agent, e.g. an oxidising agent. In the event that the iron compound is iron pentacarbonyl, the oxidising agent may be an amine oxide, for example trimethylamine N-oxide. Other related amine oxides may also be used, e.g. triethylamine N-oxide, and mixtures of such amine oxides are also suitable. The amine oxide may be suitable for decomposing the iron pentacarbonyl to form iron oxide. This may be conducted at elevated temperature, and the process may comprise heating the particles with the amine oxide to a suitable temperature. This may be for example about 120 to about 150° C. (e.g. about 120, 130, 140 or 150° C.), or it may be higher. It may be for example between about 100 and about 400° C., or about 100 to 300, 100 to 200, 200 to 400, 300 to 400 or 250 to 350° C., e.g. about 100, 150, 200, 250, 300, 350 or 400° C. The heating may be in more than one stage, each stage being at a different temperature, optionally also for a different length of time. The oxidising agent may be useful for decomposing residual iron compound that has not been decomposed by the elevated temperatures.

The process may comprise washing the particles having the magnetic granules in pores thereof. This may serve to remove residual reagents (e.g. unbound facilitation agent, solvent) as well as any magnetic granules that are sufficiently small not to be immobilised within the particles. The granules that are washed out in this step commonly have a diameter smaller than that of the windows of the particles, and are therefore readily removed. The solvent used for the washing is commonly an organic solvent, so as to be capable of readily penetrating the pores and windows of the particles and of dissolving residual reagents. It may be capable of wetting the magnetic granules so as to be capable of washing out the very small particles as described above.

The process may additionally comprise the step of immobilising a catalytic species in the pores of the hydrophobic magnetic particles. Factors affecting the stability and activity of immobilized enzymes include enzyme accessibility, support surface area, and affinity between the support and the enzyme. Enzymes may be immobilized onto a solid support by encapsulation, covalent bonding and entrapment. Encapsulation of enzymes in polymeric gels and sol-gels has limitations in that the supports usually do not possess a well-defined pore structure, negatively impacting the enzyme accessibility and the resulting catalytic activity. Although leaching might not be a prevalent problem when an enzyme is covalently bonded to a solid support, the manipulation and functionalization of enzyme may cause the protein to denature and lose its catalytic activity. Entrapping the enzymes in mesoporous silica would be ideal for enzyme immobilization since the mesoporous silica supports could be tailored with large surface areas, high pore volumes, and well-defined pore and window sizes. In particular, MCF possesses ultralarge, interconnected, cage-like mesopores that can entrap enzymes while facilitating the diffusion of substrates and products. The cage and window sizes of such materials can also be tailored to host specific enzymes, according to their molecular diameters.

The catalytic species may be immobilised in the pores by virtue of its size: the catalytic species may be unbound to the pores but may be immobilised therein by virtue of being too large to pass out of the windows of the pores. Thus they may be immobilised physically. Alternatively the catalytic species may be bound to the pore walls, for example covalently bound, optionally by means of linker groups. Commonly in the latter case the pore walls will be hydrophilic (which may be obtained from the hydrophobic magnetic particles by removing hydrophobic groups on the pore walls). Suitable catalytic species that may be covalently bound to pore walls include ring closing metathesis catalysts. These may for example be bound to the pore walls by means of urea groups. In some instances the magnetic granules in the pores of the particles may themselves have catalytic properties.

The main limitation in enzyme entrapment is the lack of affinity between the enzyme and the inorganic support surface, causing the leaching of enzyme from the support. This problem can be resolved by grafting long-chain hydrocarbons onto the surface silanol groups of siliceous supports. Such hydrophobic surface functional groups have been shown to boost the esterification activity of the immobilized lipases in organic media by 5 orders of magnitude, as compared to free enzymes in solution. The access of the substrates to the catalytic sites was promoted by the hydrophobicity of the support, and the hydrophobic interactions between the enzyme and the support prevent enzyme leaching and allow the enzyme to maintain its active conformation.

Methods of immobilizing enzymes onto hydrophobic mesoporous silica have been developed, including the conventional stirring of porous silica in an enzyme solution, and the pressure-driven method for enzyme entrapment. Low enzyme loadings and significant leaching have been reported when enzymes were immobilized onto mesoporous silica via conventional means. In contrast, the pressure-driven entrapment of enzymes in MCF has led to high enzyme loadings, high catalytic activity, improved thermal stability, and reduced enzyme leaching. In the present work, high pressure immobilisation techniques were used, however other methods may also be suitable for use with the magnetic particles of the invention.

The catalytic species may be an enzyme. This may comprise passing the catalytic species (e.g. enzyme) through the particles, optionally recycling the species through the particles, under high pressure. The high pressure may depend for example on the particle size and pore size of the particle's. It may be greater than about 10 MPa, and may be greater than about 15, 20, 25, 30, 35, 40, 45 or 50 MPa, and may be between about 10 and 50, 20 and 50, 25 and 50, 30 and 50, 40 and 50, 10 and 40, 10 and 30, 10 and 20, 25 and 40 or 25 and 30 MPa. It may be about 10, 15, 20, 25, 30, 35, 40, 45 or 50 MPa. The passing, or recycling, may be for a period of at least 30 minutes, or at least 1, 1.5, 2, 2.5, 3, 3.5, 4, 4.5 or 5 hours, and may be for between about 0.5 and 5 hours, or between about 0.5 and 2, 0.5 and 1, 1 and 5, 2 and 5 or 1 and 3 hours, and may be for about 1, 1.5, 2, 2.5, 3, 3.5, 4, 4.5 or 5 hours. It may be for sufficient time to achieve the desired loading of catalytic species. A low temperature such as 0° C. may be used during the passing or recycling, particularly when the catalytic species of limited stability. The low temperature may be between about 0 and 15° C., or between about 0 and 10, 0 and 5, 5 and 10 or 10 and 15° C., and may be about 0, 5, 10 or 15° C. The catalytic species may be a protein, a protein fragment, a saccharide, an enzyme, a DNA fragment, a peptide or a combination of two or more of these or it may be some other type of catalytic species. The catalytic species may applied to the particles in a fluid, and the fluid may be a liquid, for example an aqueous liquid. The catalytic species may be dissolved, suspended, emulsified or dispersed in the fluid. The concentration of the catalytic species in the fluid will depend on the nature of the catalytic species. The concentration may be between about 1 and 50 mg/ml, or between about 1 and 25, 1 and 10, 1 and 5, 5 and 50, 10 and 50, 25 and 50, 5 and 25 or 5 and 10 mg/ml, and may be about 1, 2, 3, 4, 5, 6, 7, 8, 9, 10, 15, 20, 25, 30, 35, 40, 45 or 50 mg/ml. The fluid may also comprise other species, for example, salts, buffers, nutrients etc. The pH of the fluid may depend on the nature of the catalytic species, and should be such that the catalytic species is stable. It may have a pH between is about 2 and 9, or between about 2 and 7, 2 and 5, 4 and 9, 7 and 9 or 4 and 7, and may have a pH of about 2, 3, 4, 5, 6, 7, 8 or 9. The catalytic species may be passed through, or recycled through, the particles at a temperature that does not denature or degrade the catalytic species and will depend on the nature of the catalytic species.

The particles having the catalytic species immobilized thereon may have greater than 50 mg catalytic species per gram of particles, or greater than 75, 100, 125, 150, 175, 200, 225, 250, 275 or 300 mg/g, and may have between about 50 and 300 mg/g, or between about 100 and 300, 150 and 300, 200 and 300, 250 and 300, 50 and 250, 50 and 100, 100 and 250 or 150 and 200 mg/g, and may have about 50, 75, 100, 125, 150, 175, 200, 225, 250, 275 or 300 mg/g. The particles may have a higher loading of catalytic species immobilized thereon than the porous support would have if it were loaded with the catalytic species under atmospheric pressure. It may be at least about 10% higher, or at least about 15, 20, 25, 30, 35, 40, 45, 50, 55, 60, 65, 70, 75, 80, 85, 90, 95 or 100% higher than the porous support would have if it were loaded with the catalytic species under atmospheric pressure. It may for example be about 15, 20, 25, 30, 35, 40, 45, 50, 55, 60, 65, 70, 75, 80, 85, 90, 95, 100, 110, 120, 130, 140 or 150% higher than the porous support would have if it were loaded with the catalytic species under atmospheric pressure. The catalytic species may be physically adsorbed into and/or onto the porous support. It may be immobilised, e.g. physically immobilised, in the pores of the porous support (i.e. the particles of the particulate material).

The invention also provides a particulate material comprising a plurality of hydrophobic magnetic particles. The particles of the material comprise mesoporous particles (optionally hydrophobic) having magnetic granules in at least some of the pores thereof. In a broader sense, the invention provides a particulate material comprising mesoporous particles having metal granules in at least some of the pores thereof. The material may be used as a catalyst, in particular a heterogeneous catalyst. It may be a magnetic material so as to facilitate separation thereof from a reaction mixture.

The mesoporous particles may be hydrophobic mesoporous silica particles. The pores may have surfaces comprising trialkylsilyl groups. The alkyl groups of the trialkylsilyl groups may be C1 to C24 alkyl or bigger than C24 alkyl. The pores may have surfaces comprising triarylsilyl, aryltrialkylsilyl or diarylalkylsilyl groups. The aryl group may be for example C6 to C12 aryl. Other suitable hydrophobic groups may be present on the silyl group. The alkyl group may be straight chain or branched chain, and may have between 1 and 24 carbon atoms, or between 1 and 18, 1 and 12, 1 and 6, 6 and 24, 12 and 24 or 6 and 18 carbon atoms, and may have 1, 2, 3, 4, 5, 6, 7, 8, 9, 10, 11, 12, 14, 16, 18, 20, 22 or 24 carbon atoms. It may comprise a cycloalkyl group such as cyclopentyl, cyclohexyl or cycloheptyl. The aryl group may be for example phenyl, biphenyl, naphthyl or some other aryl group. The aryl or alkyl group may be fluorinated or polyfluorinated or perfluorinated.

The mesoporous particles may have a structure comprising pores connected by windows, wherein the mean diameter of the windows is smaller than the mean diameter of the pores. The magnetic granules may have a mean diameter between the mean diameter of the pores and the mean diameter of the windows. The pore size and window size has been described earlier herein. The magnetic granules may have a mean diameter of about 5 to about 100 nm, or about 5 to 50, 5 to 20, 5 to 10, 10 to 100, 20 to 100, 50 to 100, 10 to 50, 10 to 20 or 20 to 50 nm, e.g. about 5, 6, 7, 8, 9, 10, 11, 12, 13, 14, 15, 20, 25, 30, 35, 40, 45, 50, 60, 70, 80, 90 or 100 nm, depending on the pore and window size of the particles. They may be present in the particles at about 1 to about 20 wt %, or about 1 to 10, 1 to 5, 1 to 2, 2 to 20, 5 to 20, 10 to 20, 2 to 10, 2 to 5 or 5 to 10 wt %, e.g. about 1, 2, 3, 4, 5, 6, 7, 8, 9, 10, 11, 12, 13, 14, 15, 16, 17, 18, 19 or 20 wt % although on occasions they may be present at an even higher loading.

The magnetic granules may comprise magnetic iron oxide, e.g. γ-Fe₂O₃.

### Examples

The entrapment of magnetic nanoparticles in the pores of hydrophobic mesocellular foams (MCF) is described. The resulting material was successfully applied towards the immobilization of Candida antartica lipase B (CALB). The activity of the resulting enzyme catalyst was tested for the acylation of 1-phenylethanol with isopropenyl acetate (see Scheme 1).

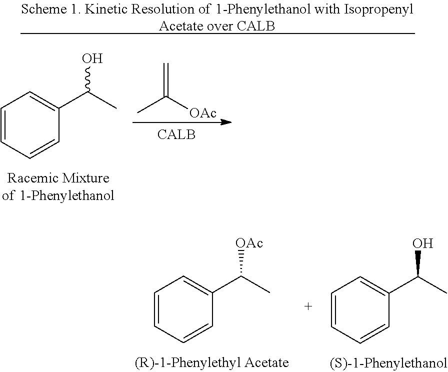

### Synthesis of Magnetic Hydrophobic MCF

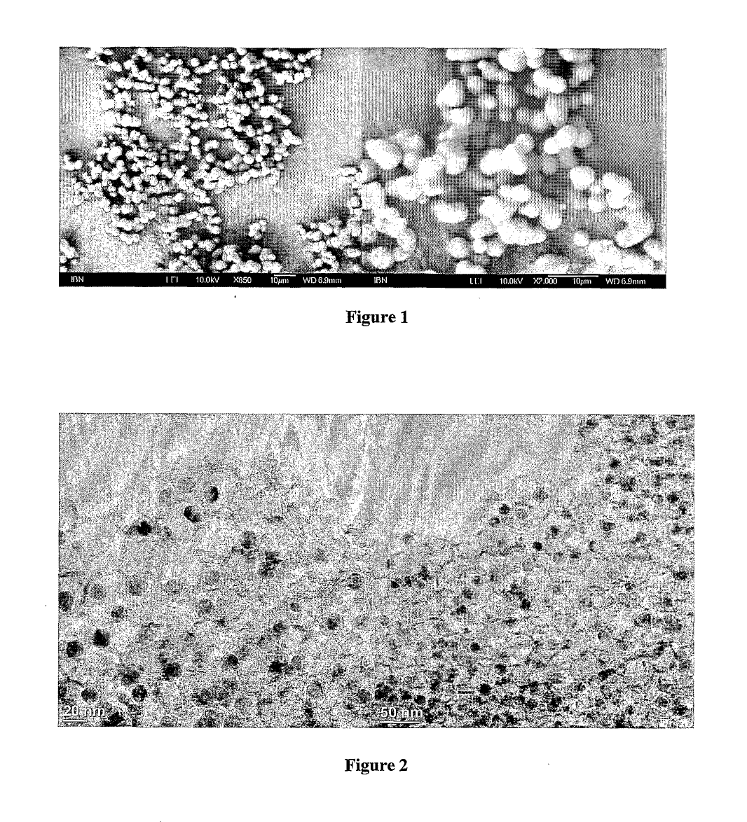

γ-Fe₂O₃ nanoparticles were entrapped in the pores of spherical hydrophobic MCF (mesocellular siliceous foam) particles. FIG. 1 illustrates the size and shape of the MCF microparticles, which were rendered hydrophobic by the reaction of the surface silanol groups with hexamethyldisilazane (HMDS), dimethylchlorooctylsilane (C₈) or dimethylchlorooctadecylsilane (C₁₈). TMS-capped MCF was obtained by vapor-phase grafting, which led to a very high loading of 1.49 mmol of TMS groups/g.

FIG. 2 shows very uniformly sized magnetic nanoparticles entrapped within the pores of MCF. During the synthesis of γ-Fe₂O₃ via thermal decomposition of Fe(CO)₅, nanoparticles were formed in the pores of hydrophobic MCF. This was aided by the presence of oleic acid surfactants around the nascent nanoparticles, which facilitated hydrophobic interactions with the hydrophobically modified silica surface. Strong interactions have previously been reported between the hydrophobic silica surface and the surfactant-capped magnetic nanoparticles. The hydrophobicity of MCF and the formation of iron-oleic acid complexes during the reaction also prevented the decomposition of Fe(CO)₅ on MCF surface. The nanoparticles were then entrapped in the pores of MCF, and subsequent washing was used to remove excess oleic acid surfactant and the smaller oleic acid-stabilized γ-Fe₂O₃ nanoparticles. In the present system, the γ-Fe₂O₃ nanoparticles could not leach from the support, as they were larger (about 15 nm) than the window size of MCF (about 10 nm). These relatively large γ-Fe₂O₃ nanoparticles facilitated the magnetic separation of the catalytic materials from the reaction medium, even at a low loading of about 5 wt % of γ-Fe₂O₃ on MCF. The loading of magnetic nanoparticles on MCF could be easily controlled by adjusting the amounts of Fe(CO)₅ and oleic acid added during the synthesis. The TMS groups in the TMS-capped MCF were easily removed by calcination, after the entrapment of magnetic nanoparticles. The calcined γ-Fe₂O₃/MCF support could then be used for the immobilization of homogeneous catalysts. xxx

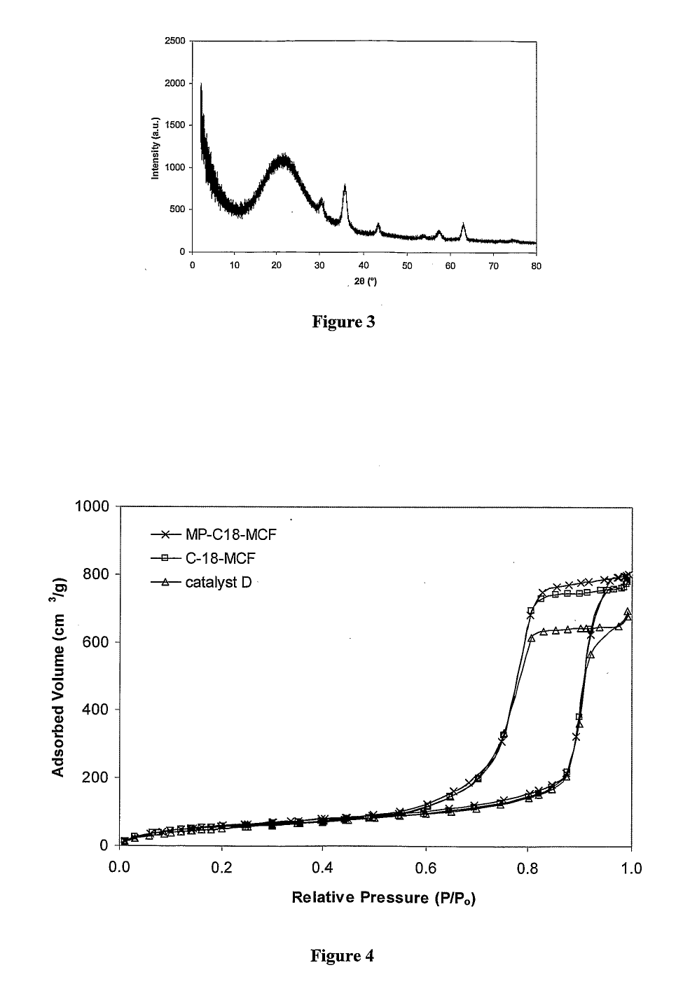

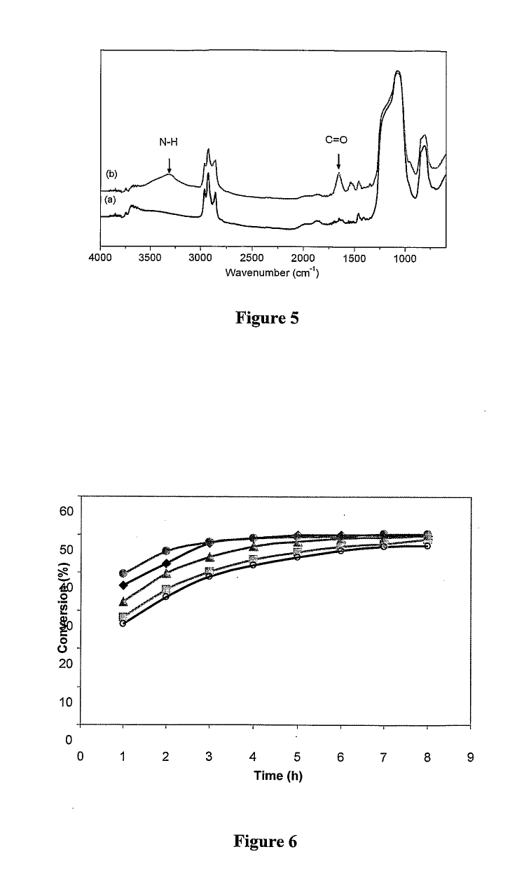

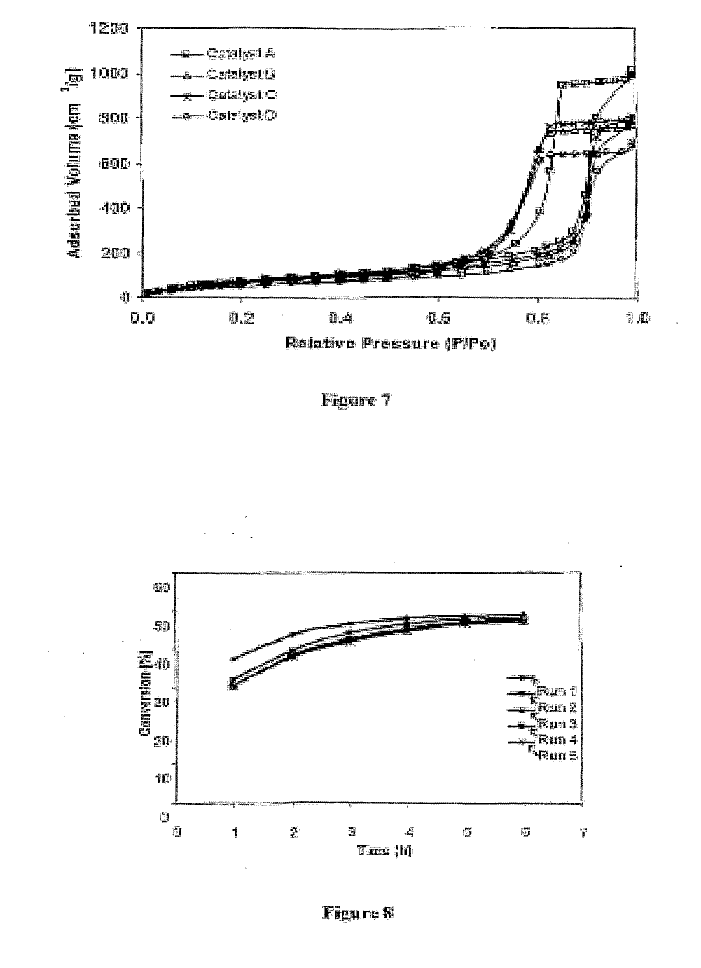

The material showed X-ray diffraction (XRD) peaks corresponding to the γ-Fe₂O₃ phase (FIG. 3). The powder XRD patterns also consisted of a broad band (2θ=11-29° associated with amorphous silica.

Previous efforts in deriving magnetically modified MCF adversely affected the total surface area and the pore size of the mesoporous material. In contrast, the BET surface area and pore volume of the present C₁₈-MCF material were only slightly reduced from 238 m²/g to 228 m²/g and from 1.22 cm³/g to 1.17 cm³/g, respectively, after the loading of about 5 wt % Fe₂O₃ nanoparticles. FIG. 4 shows the N₂ adsorption-desorption isotherm of C₁₈-MCF, before and after the incorporation of magnetic nanoparticles. The pore and window sizes of C₁₈-MCF remained almost unchanged after the entrapment of magnetic nanoparticles.

### Enzyme Immobilization

The inventors have immobilized CALB onto the magnetic hydrophobic MCF using a pressure-driven method as described in WO2006/096132, the contents of which are incorporated herein by cross reference. Enzyme loadings of 143-200 mg/g of magnetic C₈-MCF and 129 mg/g of magnetic C₁₈-MCF were achieved (Table 1). Although the enzyme loading was not as high as that reported for C₈-MCF (275 mg/g), it was still considerably higher than that attained via the conventional stirring method (92 mg/g of C₈-MCF). The lower enzyme loading on magnetic C₈-MCF as compared to C₈-MCF was likely due to the reduction of support pore volume associated with γ-Fe₂O₃ loading. The loading of magnetic nanoparticles in catalyst A was higher than that in catalyst B, which resulted in a lower enzyme loading for the former. The loadings of magnetic nanoparticles for catalysts B and C were the same; this gave rise to the same enzyme loading in these two catalysts.

TABLE 1
Enzyme Loading and Characteristics of the Catalysts

| Cata- | | Enzyme loading | Pore size | Window size |
|---|---|---|---|---|
| lyst | Support | (mg/g) | (nm) | (nm) |
| A | 5.05 wt % γ-Fe₂O₃/ | 200 | 22.3 | 8.3 |
| | C₈-MCF (5 μm) | | | |
| B | 4.62 wt % γ-Fe₂O₃/ | 143 | 22.1 | 8.4 |
| | C₈-MCF (5 μm) | | | |
| C | 4.75 wt % γ-Fe₂O₃/ | 143 | 21.4 | 8.9 |
| | C₈-MCF (2 μm) | | | |
| D | 5.43 wt % γ-Fe₂O₃/ | 129 | 20.9 | 8.6 |
| | C₁₈-MCF (5 μm) | | | |

Catalyst D was also subjected to nitrogen sorption characterization before and after enzyme immobilization. It showed only a slight reduction in BET surface area (to 210 m²/g), as compared to C₁₈-MCF (238 m²/g) and γ-Fe₂O₃/C₁₈-MCF (228 m²/g). The pore size of γ-Fe₂O₃/C₁₈-MCF was decreased slightly from 22.1 nm to 20.9 nm after enzyme loading. It indicated that the pores were not blocked by the presence of the enzymes; this was important towards facilitating the diffusion of substrates.

The entrapment of enzymes in magnetic hydrophobic MCF was characterized by photoacoustic Fourier-transform infrared (PA-FTIR) spectroscopy. The N—H stretching band (˜3300 cm⁻¹) and C═O stretching band (˜1650 cm⁻¹) were observed in the CALB/γ-Fe₂O₃/C₈-MCF catalyst (FIG. 4(b)), which were associated with the amide groups of the enzymes, confirming the enzyme incorporation. The C—H stretching band (2800-3000 cm⁻¹) was noted in the spectra of γ-Fe₂O₃/C₈-MCF before and after CALB loading; this was attributed to the hydrophobic modification of the MCF surface.

### Catalytic Activity and Selectivity in the Kinetic Resolution of 1-Phenylethanol

A fixed amount of CALB was introduced for the acylation of 1-phenylethanol with isopropenyl acetate. Catalysts A-D all achieved complete conversion (50%) of (R)-1-phenylethanol to (R)-1-phenylethylacetate in 8 h. After 1 h, catalysts D and A achieved superior conversions and catalyst C showed similar conversion, as compared to CALB entrapped in MCF. Nitrogen sorption experiments were performed on all 4 catalysts, and it was found that the pore and window sizes of all 4 catalysts were quite similar. Catalyst D (C₁₈-modified) was observed to have a higher catalytic activity than of Catalyst A (C₈-modified), and the difference could be due to the difference in the hydrophobicity of the silica support. The same trend was observed previously. Catalysts C and B have similar enzyme loading, but the former has a smaller particle size; the shorter diffusion distance for the substrate might account for catalyst C's faster reaction rate as compared to catalyst B. Catalysts A and B were similar in size and surface hydrophobicity, but the former has a higher enzyme loading, which translates to a higher catalytic activity. This was also true for catalyst A when compared to catalyst C. Catalysts A-D demonstrated higher activity than free CALB in an organic reaction mixture. This could be attributed to the good dispersion of CALB in the magnetic hydrophobic MCF, allowing the enzymes to remain unaggregated and retain its active conformation. Catalyst B has only a slight advantage over CALB in activity. This could be attributed to the leaching of enzymes from the solid support due to the weak hydrophobic interactions between the MCF surface and the enzymes, or due to enzyme degradation over time. Leaching was not a prevalent problem for catalyst A, possibly due to the crowded environment present as a result of higher enzyme loading.

The enantioselectivity of the reaction was unaffected by the entrapment of CALB in magnetic hydrophobic MCF by the pressure driven method. Over 99% ee for (R)-1-phenylethylacetate was obtained in all cases with catalysts A-D.

Recycle studies was also performed with catalyst D, as shown in FIG. 7, where the activity and the enantioselectivity of the reaction remained excellent after 5 runs. It is interesting to note that the initial activity of the catalyst dropped from 39% to 30% over 5 runs, while the drop in the final conversion of the product was minimal, from 50% to 47% over 5 runs. The initial drop of catalyst activity in the first hour for the subsequent runs might be due to the inactive, denatured state of the enzyme after the washing and drying of the catalyst, which might refold back into its tertiary structure during the course of the reaction.

### Conclusions

Novel magnetic hydrophobic MCF has been synthesized and used for enzyme entrapment. This support material incorporated maghemite nanoparticles into the siliceous MCF without disrupting the three-dimensional pore connectivity of the latter. The magnetic nanoparticles were not embedded or grafted onto the pore walls, but were rather entrapped in the pore cages like a ball in a rattle. The resulting system was effectively used as a solid support for the immobilization of CALB enzyme catalysts using a pressure driven method. The immobilized catalysts demonstrated excellent activity and enantioselectivity in the kinetic resolution of the acylation of 1-phenylethanol with isopropenyl acetate. The heterogenized catalysts could be easily separated from the reaction medium via magnetic force for reuse. This magnetic support can be broadly used for immobilization of homogeneous organometallic and organic catalysts. The entrapment method for magnetic MCF can also be employed in the synthesis of other magnetic nanoparticles that are useful for biological applications.

### Experimental Section

Materials. Spherical MCF microparticles were synthesized by modifying the literature procedure (Schmidt-Winkel, P.; Lukens, W. W., Jr.; Yang, P.; Margolese, D. I.; Lettow, J. S.; Ying, J. Y.; Stucky, G. D. Chem. Mater. 2000, 12, 686). They were rendered hydrophobic by the reaction of the surface silanol groups with HMDS, C₈ or C₁₈. The free CALB enzyme was purchased from Roche. Iron pentacarbonyl, oleic acid, trimethylamine N-oxide, anhydrous toluene, and isopropenyl acetate were obtained from Aldrich, while octyl ether and 1-phenylethanol were purchased from Fluka. Solvents such as cyclohexane, acetone and toluene were obtained from J. T. Baker (A.C.S. grade).

Synthesis of Magnetic Hydrophobic MCF. Hydrophobic MCF (2.0 g) was dispersed in a mixture of oleic acid (3.84 g, 13.68 mmol) and octylether (20 ml), and the slurry was dried under high vacuum at 50° C. overnight. The dried mixture was heated to 100° C., before Fe(CO)₅ (0.6 ml, 4.56 mmol) was injected. For catalyst A, 0.4 ml of Fe(CO)₅ was added to a mixture of oleic acid (2.56 g) and octylether (20 ml). The mixture was slowly heated to 300° C. and kept for 3 h. The colour of the mixture gradually changed from orange to black. The mixture was then cooled down to 100° C., and 1.02 g of dehydrated (CH₃)₃NO (13.68 mmol) was added. The mixture was heated to 130° C. and kept for 90 min. It was next heated to 300° C., and refluxed for 1 h before it was cooled down to room temperature. The resulting suspension was filtered and washed with cyclohexane until the filtrate was clear. The particles were then washed with methanol and acetone thoroughly, before drying under vacuum. The material was characterized by scanning electron microscopy (SEM) and transmission electron microscopy (TEM) using a JEOL JSM-7400F (5 kV) and JEOL JEM-3010 (300 kV) electron microscopes, respectively. Nitrogen sorption isotherms were obtained using a Micromeritics ASAP 2020M system, and the samples were degassed at 100° C. for 24 h before analysis. Analysis of Fe₂O₃ content in the materials was performed at the Chemical, Molecular, and Materials Analysis Centre, National University of Singapore.

Entrapment of CALB. CALB was entrapped using the pressure driven method reported previously (Han, Y.; Lee, S. S.; Ying, J. Y. Chem. Mater. 2006, 18, 643). The magnetic hydrophobic MCF was dispersed in 2-propanol and packed in a high-performance liquid chromatography (HPLC) column (100 mm×4.6 mm) using a slurry packer. 2-propanol was flushed thoroughly from the column with water, before the enzyme stock solution (0.8 mg/mL, 50 mL) was cycled through the pre-packed magnetic hydrophobic MCF column for 2 h under a pressure of 4000 psi with a slurry packer. After enzyme loading, a PBS buffer solution and pure water were sequentially flowed through the column at a pressure of 2000 psi for 30 min before the water flow direction was reversed to wash the column at a pressure of 2000 psi for 30 min. The enzyme-loaded magnetic hydrophobic MCF was then collected from the column, and washed with water and toluene, before drying under vacuum. It was then stored at 4° C. before use.

Four catalysts were prepared with magnetic hydrophobic MCF microparticles with different particle sizes, hydrophobic surface treatments, γ-Fe₂O₃ loadings, and CALB loadings. The enzyme loadings in the catalysts were determined by C, H and N analyses using a CE440 CHN analyzer. PA-FTIR spectra were recorded on a Digilab FTS 7000 FTIR spectrometer equipped with a MTEC-300 photoacoustic detector.

Catalytic Reaction. The catalysts were tested for the kinetic resolution of the acylation reaction of 1-phenylethanol with isopropenyl acetate. (R)-1-phenylethanol would selectively react with isopropenyl acetate in the presence of the active enzyme, leaving (S)-1-phenylethanol unreacted. In a typical reaction, an appropriate amount of heterogenized catalytic material containing a total CALB loading of 2 mg would be dispersed in 1.5 mL of dry toluene. An aliquot (1.5 mL) of 1-phenylethanol stock solution in toluene (1.44 mmol/mL) was added at room temperature, followed by isopropenyl acetate. A small aliquot was withdrawn from the reaction mixture every hour, and the reaction was monitored with gas chromatography until complete conversion was obtained, as indicated by the disappearance of the (R)-1-phenylethanol peak. At the end of 8 h, the reaction mixture was analyzed by HPLC for enantiomeric excess (% ee) using a chiral Daicel® OD-H column.

Recyclability of Catalyst D. In a typical recycling experiment, an appropriate amount of catalyst D containing a total CALB loading of 10 mg was dispersed in 7.5 mL of dry toluene. 7.5 mL of 1-phenylethanol in toluene (1.44 mmol/mL, total amount equivalent to 10.8 mmol) was added at room temperature. 17.4 mmol of isopropenyl acetate was then added, and a small aliquot of the reaction mixture was withdrawn every hour. The reaction was monitored with gas chromatography (GC) over 6 h. At the end of 6 h, the reaction mixture was analysed by HPLC for enantiomeric excess (ee) using a Daicel® OD-H column. The catalyst was filtered and washed 6 times with 30 mL of toluene, and dried under vacuum at room temperature for 40 h, before being reweighed and used for subsequent runs.

1. A process for making a particulate material comprising mesoporous particles having granules of a metal containing species in at least some of the pores thereof, said process comprising:
allowing a compound of the metal to enter pores of hydrophobic mesoporous particles, said compound being thermally decomposable at a decomposition temperature to form a metal containing species and said particles being substantially thermally stable at said decomposition temperature; and
heating the hydrophobic mesoporous particles having the compound in the pores thereof to the decomposition temperature so as to decompose the compound and to form the mesoporous particles having granules of the metal containing species in at least some of the pores thereof.

2. The process of claim 1, said particulate material comprising hydrophobic magnetic particles, said process comprising:
allowing an iron compound to enter pores of hydrophobic mesoporous particles, said iron compound being thermally decomposable at a decomposition temperature and said particles being substantially thermally stable at said decomposition temperature; and
heating the hydrophobic mesoporous particles having the iron compound in the pores thereof to the decomposition temperature so as to decompose the iron compound and to form the hydrophobic magnetic particles having magnetic granules in at least some of the pores.

3. The process of claim 1 or claim 2 comprising the step of allowing a facilitation agent to enter the pores of the mesoporous particles prior to allowing the iron to enter said pores.

4. The process of claim 3 wherein the facilitation agent is a carboxylic acid.

5. The process of any one of claims 1 to 4 wherein the iron compound is an iron carbonyl complex.

6. The process of claim 5 wherein the iron compound is iron pentacarbonyl.

7. The process of any one of claims 1 to 6 wherein the decomposition temperature is about 250 to about 350° C.

8. The process of any one of claims 1 to 7 comprising the additional steps of cooling the particles and treating the cooled particles with an oxidising agent.

9. The process of claim 8 wherein the iron compound is iron pentacarbonyl and the oxidising agent is trimethylamine N-oxide.

10. The process of any one of claims 1 to 9 wherein the granules comprise magnetic γ-Fe₂O₃.

11. The process of any one of claims 1 to 10 wherein the mesoporous particles are hydrophobic mesoporous silica.

12. The process of claim 11 comprising reacting mesoporous silica particles with a hydrophobing agent so as to produce the hydrophobic mesoporous silica.

13. The process of any one of claims 1 to 12 wherein surfaces of the pores of the mesoporous particles comprise trimethylsilyl groups, dimethyloctylsilyl groups, dimethyloctadecylsilyl groups or a mixture of any two or all of these.

14. The process of any one of claims 1 to 13 additionally comprising the step of immobilising a catalytic species in the pores of the particles.

15. The process of claim 14 wherein the catalytic species is an enzyme.

16. The process of claim 15 wherein the particles are hydrophobic and the step of immobilising the enzyme comprises passing a fluid comprising the enzyme through the hydrophobic particles under high pressure.

17. The process of claim 16 wherein the pressure is between about 25 and about 50 MPa.

18. The process of claim 16 or claim 17 wherein the fluid is an aqueous liquid.

19. A particulate material comprising a plurality of magnetic particles, said particles comprising mesoporous particles having magnetic granules in at least some of the pores thereof.

20. The particulate material of claim 19 wherein the magnetic particles and the mesoporous particles are both hydrophobic.

21. The particulate material of claim 19 or claim 20 wherein the mesoporous particles are mesoporous silica particles.

22. The particulate material of any one of claims 19 to 21 wherein pores of the mesoporous particles have surfaces comprising trialkylsilyl groups.

23. The particulate material of any one of claims 19 to 22 wherein the mesoporous particles have a structure comprising pores connected by windows, wherein the mean diameter of the windows is smaller than the mean diameter of the pores.

24. The particulate material of claim 23 wherein the magnetic granules have a mean diameter between the mean diameter of the pores and the mean diameter of the windows.

25. The particulate material of any one of claims 19 to 24 wherein the magnetic granules comprise magnetic γ-Fe₂O₃.

26. The particulate material of any one of claims 19 to 25, said particulate material having a catalytic species immobilised in the pores of the particles.

27. The particulate material of claim 26 wherein the catalytic species is an enzyme.

28. The particulate material of claim 27 wherein the diameter of the enzyme is between the mean diameter of the pores and the mean diameter of the windows.

29. Use of a particulate material according to any one of 19 to 28 as a catalyst.

30. A particulate material according to any one of claims 19 to 28 when used as a catalyst.

31. A method for converting a starting material to a product, said method comprising exposing the starting material to a particulate material according to any one of claims 19 to 28, wherein the catalytic species is capable of catalysing the conversion of the starting material to the product.

32. The method of claim 31 wherein the starting material is dissolved in a solvent during the step of exposing said starting material to said particulate material.

33. The method of claim 32 wherein the solvent is a non-polar solvent.

34. The method of any one of claims 31 to 33 comprising the step of separating the particulate material from the starting material and the product.

35. The method of claim 34 wherein the step of separating comprises exposing the particulate material to a magnetic field.

36. The method of any one of claims 31 to 35 comprising the step of reusing the particulate material as a catalyst in a subsequent reaction.

37. The method of claim 36 wherein the subsequent reaction converts the starting material to the product, and the yield of the product of the subsequent reaction is at least 90% of the yield of the product from the previous reaction.

38. A particulate material comprising a plurality of particles, said particles comprising mesoporous particles having granules of a metal containing species in at least some of the pores thereof.

39. The particulate material of claim 38 wherein the granules of the metal containing species and the mesoporous particles are both hydrophobic.

40. The particulate material of claim 38 or claim 39 wherein the mesoporous particles are mesoporous silica particles.

41. The particulate material of any one of claims 38 to 40 wherein pores of the mesoporous particles have surfaces comprising trialkylsilyl groups.

42. The particulate material of any one of claims 38 to 41 wherein the mesoporous particles have a structure comprising pores connected by windows, wherein the mean diameter of the windows is smaller than the mean diameter of the pores.

43. The particulate material of claim 38 wherein the granules of the metal containing species have a mean diameter between the mean diameter of the pores and the mean diameter of the windows.

44. The particulate material of any one of claims 38 to 43 wherein the granules of the metal containing species comprise a transition metal.

45. The particulate material of any one of claims 38 to 44, said particulate material having a catalytic species immobilised in the pores of the particles.

46. The particulate material of claim 45 wherein the catalytic species is an enzyme.

47. The particulate material of claim 46 wherein the diameter of the enzyme is between the mean diameter of the pores and the mean diameter of the windows.

48. The particulate material according to any one of claims 38 to 47 further comprising magnetic granules.

49. The particulate material of claim 48 wherein the magnetic granules comprise magnetic γ-Fe₂O₃.

50. Use of a particulate material according to any one of 38 to 49 as a catalyst.

51. A particulate material according to any one of claims 38 to 49 when used as a catalyst.
